(19) United States
(12) Patent Application Publication
(10) Pub. No.: US 2023/0181544 A1
(43) Pub. Date: Jun. 15, 2023

(54) DOSING REGIMENS FOR USE IN TREATING RELAPSED AND REFRACTORY MULTIPLE MYELOMA WITH VENETOCLAX

(71) Applicant: ABBVIE INC., North Chicago, IL (US)

(72) Inventors: Orlando F. BUENO, North Chicago, IL (US); Yan LUO, Lake Forest, IL (US); Laura Leanne FLEMING, Park Ridge, IL (US); Mohamed BADAWI, North Chicago, IL (US); Ahmed Hamed SALEM, Libertyville, IL (US)

(73) Assignee: ABBVIE INC., North Chicago, IL (US)

(21) Appl. No.: 18/077,929

(22) Filed: Dec. 8, 2022

Related U.S. Application Data
(60) Provisional application No. 63/288,466, filed on Dec. 10, 2021.

(51) Int. Cl. A61K 31/437; C07K 16/28; A61K 31/573; A61K 45/06; A61K 9/00; A61P 35/00

(52) U.S. Cl. CPC A61K 31/437; C07K 16/2896; A61K 31/573; A61K 45/06; A61K 9/0019; A61K 9/0056; A61P 35/00

(57) ABSTRACT

The invention described herein relates to dosing methods for treating relapsed and refractory multiple myeloma (RRMM) in a human subject comprising administering to the subject venetoclax in combination with daratumumab and dexamethasone.

### CROSS-REFERENCE TO RELATED APPLICATIONS

This application claims the benefit of U.S. Provisional Application No. 63/288,466, filed Dec. 10, 2021, the disclosure of which is incorporated by reference herein in its entirety.

### FIELD OF THE INVENTION

This invention relates to dosing methods for treating relapsed and refractory multiple myeloma (RRMM) in a human subject comprising administering to the subject venetoclax in combination with daratumumab and dexamethasone. Also, provided herein is venetoclax in combination with daratumumab and dexamethasone for use in methods of treating relapsed or refractory multiple myeloma.

### BACKGROUND OF THE INVENTION

Multiple myeloma (MM) is a rare cancer caused by abnormal survival of plasma cells (blood cells). In the relapsed/refractory (RR) setting, MM becomes increasingly aggressive, and remission durations often decrease with each subsequent regimen. Several therapeutic agents with unique mechanisms of action have recently been approved for RRMM.

Venetoclax is a highly selective, potent, oral Bcl-2 inhibitor that promotes apoptosis by direct, high-affinity binding to its prosurvival target, Bcl-2. In studies, venetoclax as a monotherapy and in combination with other agents, has demonstrated meaningful clinical activity in RRMM. (Kumar et al. Blood. 2017; 130(22):2401-2409 and Moreau et al. Blood. 2017; 130(22):2392-2400).

However, there remains a need for improved venetoclax combination regimens to treat RRMM.

### BRIEF SUMMARY OF THE INVENTION

One aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment. In some embodiments, the human subject has t(11;14) multiple myeloma.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, and the dose of 400 mg of venetoclax once daily remains unchanged during treatment, wherein, during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; and wherein the human subject has t(11;14) multiple myeloma.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a third, fourth, fifth, and sixth cycle, each of the cycles consisting of 28 days, 400 mg of venetoclax is administered on days 1-28, and 1800 mg of daratumumab is subcutaneously administered on days 1 and 15, and 40 mg of dexamethasone is orally or intravenously administered on days 1, 8, 15, and 22; and wherein the human subject has t(11;14) multiple myeloma.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22; and wherein the human subject has t(11;14) multiple myeloma.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a stringent complete response (sCR).

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a complete response (CR).

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a very good partial response (VGPR).

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a partial response (PR).

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in minimal residual disease (MRD).

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in MRD<10⁻⁴.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject and wherein the dose of 400 mg of venetoclax once daily remains unchanged during treatment, wherein the method results in MRD<10⁻⁵.

Another aspect pertains to a method for treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in MRD<10⁻⁶.

Another aspect pertains to a method for treating a human subject having t(11;14) relapsed or refractory multiple myeloma, comprising administering to the subject venetoclax in combination with daratumumab and dexamethasone, wherein 400 mg of venetoclax is administered once daily, wherein the dose remains unchanged at 400 mg of venetoclax once daily during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle of 28 days, 400 mg of venetoclax is administered on days 1-28, daratumumab is administered subcutaneously at a dose of 1800 mg on days 1 and 15, and dexamethasone is administered orally or intravenously at a dose of 40 mg on days 1, 8, 15, and 22; and wherein during a seventh cycle or longer of 28 days, 400 mg of venetoclax is administered on days 1-28, daratumumab is administered subcutaneously at a dose of 1800 mg on day 1, and dexamethasone is administered orally or intravenously at a dose of 40 mg on days 1, 8, 15, and 22.

### BRIEF DESCRIPTION OF THE FIGURES

FIG. 1 shows overall response rate in Example 1, according to one embodiment.

FIG. 2 shows minimal residual disease in Example 1, according to one embodiment.

FIG. 3 shows adverse events in Example 1, according to one embodiment.

FIG. 4 shows most common hematologic adverse events in Example 1, according to one embodiment.

FIG. 5 shows details of infections in Example 1, according to one embodiment.

FIG. 6 shows detailed responses in Example 1, according to one embodiment.

### DETAILED DESCRIPTION OF THE INVENTION

In certain embodiments, the methods relate to the treatment of a human subject having relapsed and refractory multiple myeloma (RRMM) comprising administering to the subject venetoclax in combination with daratumumab and dexamethasone.

In certain embodiments, the methods relate to the treatment of a human subject having relapsed multiple myeloma comprising administering to the subject venetoclax in combination with daratumumab and dexamethasone.

In certain embodiments, the methods relate to the treatment of a human subject having refractory multiple myeloma comprising administering to the subject venetoclax in combination with daratumumab and dexamethasone.

In certain embodiments, the methods relate to the treatment of a human subject having t(11;14) relapsed and refractory multiple myeloma (RRMM) comprising administering to the subject venetoclax in combination with daratumumab and dexamethasone, e.g., according to a dosing described herein.

In certain embodiments, the methods relate to the treatment of a human subject having t(11;14) relapsed multiple myeloma comprising administering to the subject venetoclax in combination with daratumumab and dexamethasone.

In certain embodiments, the methods relate to the treatment of a human subject having t(11;14) refractory multiple myeloma comprising administering to the subject venetoclax in combination with daratumumab and dexamethasone.

In certain embodiments, venetoclax is used in combination with daratumumab and dexamethasone for the methods disclosed herein, for treating relapsed and/or refractory multiple myeloma, for example, t(11;14) relapsed and/or refractory multiple myeloma

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment.

In certain embodiments, the methods relate to the treatment of a human subject having relapsed multiple myeloma, comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment.

In certain embodiments, the methods relate to the treatment of a human subject having refractory multiple myeloma, comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject. In some embodiments, the dose of 400 mg of venetoclax once daily remains unchanged during treatment. In some embodiments, the human subject has t(11;14) multiple myeloma.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22.

In certain embodiments, the methods relate to the treatment of a human subject having relapsed multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22.

In certain embodiments, the methods relate to the treatment of a human subject having refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22, and wherein the human subject has t(11;14) multiple myeloma.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22.

In certain embodiments, the methods relate to the treatment of a human subject having relapsed multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22.

In certain embodiments, the methods relate to the treatment of a human subject having refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein, during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22, and wherein the human subject has t(11;14) multiple myeloma.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having relapsed multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22; and wherein the human subject has t(11;14) multiple myeloma.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having relapsed multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein, during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein, during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; and wherein, during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22; and wherein the human subject has t(11;14) multiple myeloma.

In certain embodiments, the methods relate to the treatment of a human subject having t(11;14) relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to the treatment of a human subject having t(11;14) relapsed or refractory multiple myeloma (RRMM), comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment; wherein during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22; wherein during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22; and wherein during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a complete response (CR).

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a very good partial response (VGPR).

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in a partial response (PR).

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in minimal residual disease (MRD).

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in MRD<10⁻⁴.

In certain embodiments, the methods relate to treating a human subject having relapsed or refractory multiple myeloma, comprising administering to the subject venetoclax in combination with daratumumab and dexamethasone, wherein a dose of 400 mg of venetoclax is orally administered once daily to the subject, wherein the dose of 400 mg of venetoclax once daily remains unchanged during treatment, and wherein the method results in MRD<10⁻⁵.

DARZALEX® (daratumumab) injection is a colorless to pale yellow, preservative-free solution available as a 100 mg/5 mL solution in a single-dose vial and a 400 mg/20 mL solution in a single-dose vial for dilution.

Dexamethasone is designated chemically as 9-fluoro-11β,17,21-trihydroxy-16α-methylpregna-1,4-diene,3,20-dione.

Dexamethasone Tablets USP are available for oral administration containing either 0.5 mg, 0.75 mg, 1 mg, 1.5 mg, 2 mg, 4 mg or 6 mg of dexamethasone USP.

Dexamethasone Oral Solution USP is formulated for oral administration containing 0.5 mg per 5 mL of dexamethasone USP.

Dexamethasone is designated chemically as 9-fluoro-11β,17,21-trihydroxy-16α-methylpregna-1,4-diene,3,20-dione.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent documents incorporated by reference contradict the disclosure contained in the specification; the specification will supersede any contradictory material.

So that the disclosure may be more readily understood, select terms are defined below.

Abbreviations and Definitions

As used in the specification and the appended claims, unless specified to the contrary, the following terms have the meaning indicated:

The terms “treat”, “treating” and “treatment” refer to a method of alleviating or abrogating a disease and/or its attendant symptoms and encompass treatment of the disease resulting in stringent complete response, complete response, very good partial response, partial response, and/or minimal residual disease.

The term “subject” means a human.

The terms “patient” and “subject” are used herein interchangeably.

The term “IV” means intravenous.

The term “R/R” means relapsed/refractory.

The term “sCR” means stringent complete response as determined by the guidelines established by the International Myeloma Working Group (IMWG).

Guidelines established by the IMWG can be found, for example, in Kumar, S. et al., “International Myeloma Working Group consensus criteria for response and minimal residual disease assessment in multiple myeloma,” Lancet Oncol. 17, e328-e346 (2016).

The term “CR” means complete response as determined by the guidelines established by the International Myeloma Working Group (IMWG).

The term “VGPR” means very good partial response as determined by the guidelines established by the International Myeloma Working Group (IMWG).

The term “PR” means partial response as determined by the guidelines established by the International Myeloma Working Group (IMWG).

The term “MRD” means minimal residual disease as determined by the guidelines established by the International Myeloma Working Group (IMWG).

The term “ORR” means overall response rate as determined by the guidelines established by the International Myeloma Working Group (IMWG).

The term “RRMM” means relapsed and refractory multiple myeloma.

The term “ECOG” means Eastern Cooperative Oncology Group.

The term “PI” means proteosome inhibitor.

The term “IMID” means immunomodulatory imide drug, for example, lenalidomide, pomalidomide, and thalidomide.

The term “SCT” means stem cell transplant.

The term “SC” means subcutaneously.

The term “Ven” means venetoclax.

The term “D” means daratumumab.

The term “d” means dexamethasone.

The term “Ab” means antibody.

The term “V” means bortezomib.

The term “ISS” means international staging system.

The term “FISH” means fluorescence in situ hybridization.

The term “TEAE” means treatment emergent adverse event.

“Venetoclax” is 4-{4-[(4′-chloro-5,5-dimethyl-3,4,5,6-tetrahydro[1,1′-biphenyl]-2-yl)methyl]piperazin-1-yl}-N-(3-nitro-4-{[(oxan-4-yl)methyl]amino}benzene-1-sulfonyl)-2-[(1H-pyrrolo[2,3-b]pyridin-5-yl)oxy]benzamide. The following is a chemical structure for venetoclax:

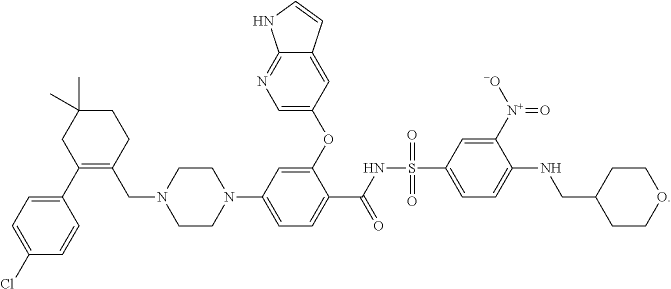

“DARZALEX®” is daratumumab, a CD38-directed cytolytic antibody.

Dexamethasone is 9-fluoro-11β,17,21-trihydroxy-16α-methylpregna-1,4-diene,3,20-dione. The following is a chemical structure for dexamethasone:

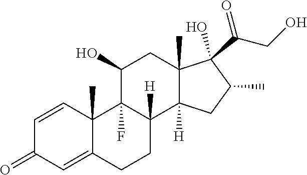

The term “remains unchanged during treatment” means that the dose administered to the patient on the first day of treatment is also administered on subsequent days of treatment until treatment is stopped or unacceptable toxicity occurs.

The use of the terms “a” and “an” and “the” and similar referents in the context of describing the invention (especially in the context of the following claims) are to be construed to cover both the singular and the plural, unless otherwise indicated herein or clearly contradicted by context.

In order that the invention described herein may be more fully understood, the following example is set forth.

### Example 1

A Randomized Open-label Expansion Study of Venetoclax (Ven), Daratumumab (D), and Dexamethasone (d) in Patients with t(11;14) RRMM

The primary objective of the study was to compare and further evaluate the safety and preliminary efficacy of VenDd at 400 mg and 800 mg Ven dose levels with daratumumab (D), and dexamethasone (d).

Key Inclusion Criteria: Adult (≥18 years) with t(11:14) RRMM, measurable disease, ECOG performance status≤2, Non-refractory to PIs, Non-refractory to anti-CD38 Ab, ≥1 prior line of therapy, and adequate hematologic, renal and hepatic function.

Key Exclusion Criteria: Prior treatment with venetoclax (or other Bc1-2 inhibitors), peripheral neuropathy≥grade 3 or ≥grade 2 with pain within 2 weeks prior to first dose, autologous SCT within 12 weeks prior to first dose; previous allogenic SCT, prior anti-CD38 Ab treatment within 6 months prior to first dose of study drug, and prior anti-myeloma treatment 2-6 weeks before study entry depending on modality. Table 1 shows study dosing.

TABLE 1
VenDd

| | Ven (400/800 mg) | D (1800 mg) | d (40 mg^{a}) |
|---|---|---|---|
| Cycle | oral | SC | oral or IV |
| 1-2 | Once daily | Days 1, 8, 15, 22 | Weekly |
| (28 days) | | | |
| 3-6 | Once daily | Days 1 and 15 | Weekly |
| (28 days) | | | |
| 7+ | Once daily | Day 1 | Weekly |
| (28 days) | | | |

| | DVd | | |
|---|---|---|---|
| | V (1.3 mg/m²) | D (1800 mg) | d (20 mg) |
| Cycle | SC or IV | SC | oral or IV |
| 1-3 | Days 1, 4, 8, 11 | Day 1, 8, 15 | Day 1, 2, 4, 5, 8, 9, |
| (28 days) | | | 11, 12, and 15^{b} |
| 4-8 | Days 1, 4, 8, 11 | Day 1 | Day 1, 2, 4, 5, 8, 9, |
| (28 days) | | | 11, and 12 |
| 9+ | — | Day 1 | Day 1^{c} |
| (21 days) | | | |

Ven, venetoclax;
Ven400, venetoclax 400 mg;
Ven800, venetoclax 800 mg;
D, daratumumab; d, dexamethasone;
Ab, antibody;
V, bortezomib;
RRMM, relapsed refractory multiple myeloma;
PI, proteosome inhibitor;
IMID, immunomodulatory agent;
SC, subcutaneous;
IV, intravenous.
Footnotes to Table 1:
^{a}A split dose of 20 mg on day 1 and 20 mg on day 2 of each week was allowed at the discretion of investigators/local guidance.
^{b}Also allowed on day 16 at the discretion of investigators/local guidance.
^{c}Also allowed on day 2 at the discretion of investigators/local guidance.

Table 2 shows patient characteristics were balanced between the two VenDd arms.

TABLE 2

| | Ven400Dd | | Ven800Dd | | DVd | |
|---|---|---|---|---|---|---|
| Characteristic | n = 15 | | n = 7 | | n = 19 | |
| Median age, | 58.0 | (31-88) | 57.0 | (53-82) | 70.0 | (51-80) |
| years (range) | | | | | | |
| ISS stage, n (%) | | | | | | |
| I | 6 | (40.0) | 4 | (57.1) | 5 | (26.3) |
| II | 3 | (20.0) | 1 | (14.3) | 4 | (21.0) |
| III | 1 | (6.7) | 1 | (14.3) | 5 | (26.3) |
| Unknown/missing | 5 | (33.3) | 1 | (14.3) | 5 | (26.3) |
| ECOG performance | | | | | | |
| status, n (%) | | | | | | |
| 0 | 8 | (53.3) | 6 | (85.7) | 6 | (31.6) |
| 1 | 7 | (46.7) | 1 | (14.3) | 11 | (57.9) |
| ≥2 | 0 | | 0 | | 2 | (10.5) |
| Cytogenetic | | | | | | |
| abnormality risk by | | | | | | |
| FISH, n (%) | | | | | | |
| High^{a} | 2 | (13.3) | 1 | (14.3) | 2 | (10.5) |
| Standard^{b} | 10 | (66.7) | 4 | (57.1) | 11 | (57.9) |
| Unknown/missing^{c} | 3 | (20.0) | 2 | (28.6) | 6 | (31.6) |
| No. of prior lines of | 1.0 | (1-6) | 1.0 | (1-4) | 2.0 | (1-4) |
| therapy, median | | | | | | |
| (range) | | | | | | |
| 1 | 9 | (60.0) | 4 | (57.1) | 4 | (21.1) |
| 2 | 3 | (20.0) | 1 | (14.3) | 12 | (63.2) |
| 3 | 2 | (13.3) | 1 | (14.3) | 2 | (10.5) |
| >3 | 1 | (6.7) | 1 | (14.3) | 1 | (5.3) |
| Stem cell | 7 | (46.7) | 2 | (28.6) | 8 | (42.1) |
| transplantation, n (%) | | | | | | |
| Prior PI, n (%) | 14 | (93.3) | 7 | (100) | 17 | (89.5) |
| Prior IMiD, n (%) | 14 | (93.3) | 7 | (100) | 19 | (100) |

| | | | | | |
|---|---|---|---|---|---|
| Prior anti-CD38 | | | | | |
| monoclonal antibodies, | | | | | |
| n (%) | | | | | |
| | 0 | 1 | (14.3) | 1 | (5.3) |

Ven, venetoclax;
Ven400, venetoclax 400 mg;
Ven800, venetoclax 800 mg;
D, daratumumab;
d, dexamethasone;
V, bortezomib;
FISH, fluorescence in situ hybridization;
PI, proteosome inhibitor;
IMID, immunomodulatory imide drug; ECOG, Eastern Cooperative Oncology Group.
Footnotes to Table 2:
^{a}t(4; 14) or t(14; 16) or del(17p).
^{b}No high-risk cytogenetics.
^{c}Sample was tested but results were inconclusive.

Table 3 shows there were no treatment emergent deaths. The median time on Ven for Ven400Dd and Ven800Dd were 6.0 months (range 0.2-16.5) and 8.5 months (range 1.8-15.9), respectively. The median time on study treatment for DVd was 3.9 months (0.5-14.8) based on exposure to D. One death occurred in the DVd arm 3 months after study treatment due to progressive disease after subsequent therapy.

TABLE 3

| | Ven400Dd | | Ven800Dd | DVd | |
|---|---|---|---|---|---|
| | n = 15 | | n = 7 | n = 18^{a} | |
| Active on Ven | 14 | (93) | 7 (100) | 11 | (61) |
| (or control), n (%) | | | | | |
| Discontinued Ven | 1 | (6.7) | 0 | 7 | (38.9) |
| (or control), n (%) | | | | | |
| Primary reason for d/c | | | | | |
| of study drug, n (%) | | | | | |
| Disease progression | 1 | (6.7) | 0 | 6 | (33.3) |

| | | | | |
|---|---|---|---|---|
| Other | 0 | 0 | 1 | (5.6) |
| Any death, n (%) | 0 | 0 | 1 | (5.6) |

| | | | |
|---|---|---|---|
| Treatment-emergent death, n (%) | 0 | 0 | 0 |

Ven, venetoclax;
Ven400, venetoclax 400 mg;
Ven800, venetoclax 800 mg;
D, daratumumab;
d, dexamethasone;
V, bortezomib.
Footnote to Table 3:
^{a}One patient withdrew consent prior to first dose and is included in the ITT population but not the safety analysis population; active and discontinuations for DVd is based on D.

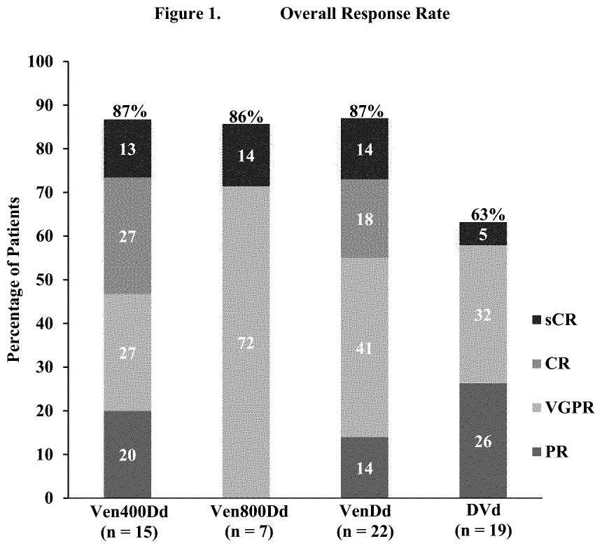

Overall Response Rate (ORR) is shown in FIG. 1. ORR was similar for patients treated with Ven400Dd and Ven800Dd.

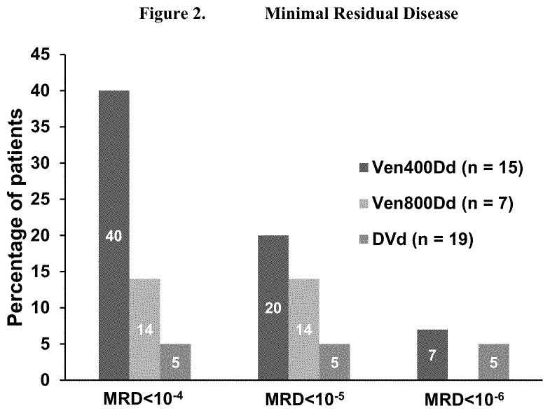

Minimal Residual Disease (MRD) rates are shown in FIG. 2. Among the 22 patients treated, for those patients treated with Ven400Dd, 40% showed MRD<10⁻⁴, 20% showed MRD<10⁻⁵, and 7% showed MRD<10⁻⁶, while for those patients treated with Ven800Dd, 14% showed MRD<10⁻⁴, 14% showed MRD<10⁻⁵, and 0% showed MRD<10⁻⁶.

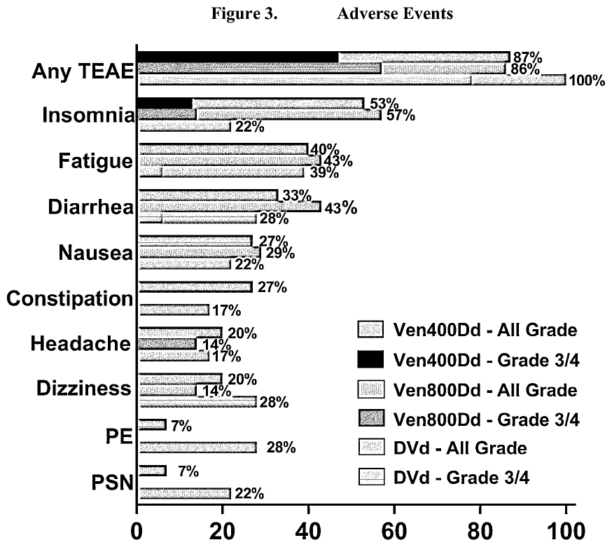

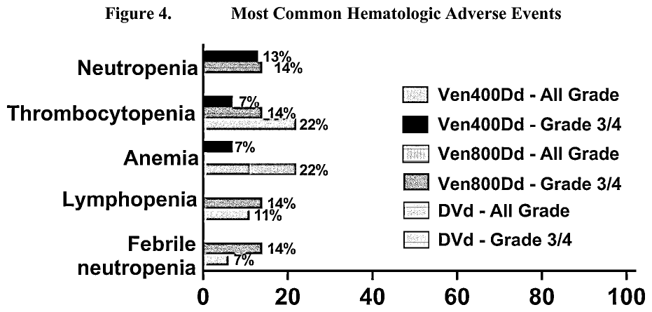

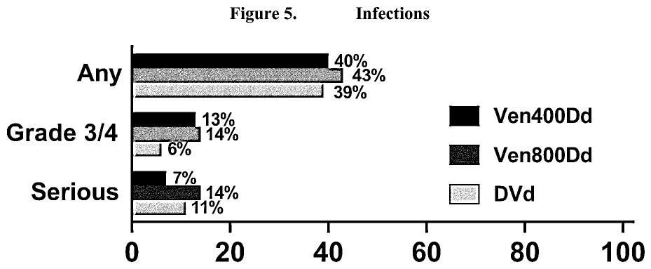

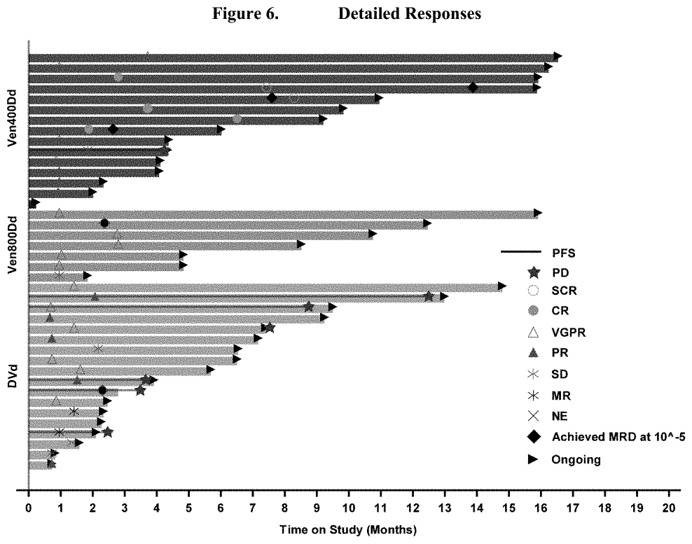

Adverse Events (AEs) are shown in FIG. 3, Most Common Hematologic Adverse Events are shown in FIG. 4, and Infections are shown in FIG. 5. Detailed responses for each patient in the study are shown in FIG. 6.

It is understood that the foregoing detailed description and accompanying examples are merely illustrative and are not to be taken as limitations upon the scope of the present disclosure.

We claim:

1. A method for treating a human subject having relapsed or refractory multiple myeloma, comprising administering to the subject venetoclax in combination with daratumumab and dexamethasone, wherein a dose of 400 mg of venetoclax is orally administered once daily to the subject and wherein the dose of 400 mg of venetoclax once daily remains unchanged during treatment.

2. The method of claim 1, wherein the human subject has t(11;14)multiple myeloma.

3. The method of claim 1, wherein, during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22.

4. The method of claim 3, wherein the human subject has t(11;14) multiple myeloma.

5. The method of claim 1, wherein, during a third, fourth, fifth, and sixth cycle, each of the cycles consisting of 28 days, 400 mg of venetoclax is administered on days 1-28, 1800 mg of daratumumab is subcutaneously administered on days 1 and 15, and 40 mg of dexamethasone is orally or intravenously administered on days 1, 8, 15, and 22.

6. The method of claim 5, wherein the human subject has t(11;14) multiple myeloma.

7. The method of claim 1, wherein, during a seventh cycle or subsequent cycle of 28 days, venetoclax is administered on days 1-28, daratumumab is administered subcutaneously at a dose of 1800 mg on day 1, and dexamethasone is administered orally or intravenously at a dose of 40 mg on days 1, 8, 15, and 22.

8. The method of claim 7, wherein the human subject has t(11;14) multiple myeloma.

9. The method of claim 1, wherein the method results in a stringent complete response (sCR).

10. The method of claim 1, wherein the method results in a complete response (CR).

11. The method of claim 1, wherein the method results in a very good partial response (VGPR).

12. The method of claim 1, wherein the method results in a partial response (PR).

13. The method of claim 1, wherein the method results in minimal residual disease (MRD).

14. The method of claim 1, wherein the method results in MRD<10⁻⁴.

15. The method of claim 1, wherein the method results in MRD<10⁻⁵.

16. The method of claim 1, wherein the method results in MRD<10⁻⁶.

17. A method for treating a human subject having t(11;14) relapsed or refractory multiple myeloma, comprising administering to the subject venetoclax in combination with daratumumab and dexamethasone, wherein 400 mg of venetoclax is administered once daily and wherein the dose remains unchanged at 400 mg of venetoclax once daily during treatment; wherein:
during a first and a second treatment cycle, each of the cycles consisting of 28 days, 400 mg of venetoclax is orally administered on days 1-28, 1800 mg of daratumumab is subcutaneously administered on days 1, 8, 15, and 22, and 40 mg of dexamethasone is orally or intravenously administered on days 1, 8, 15, and 22;
during a third, fourth, fifth, and sixth cycle of 28 days, 400 mg of venetoclax is administered on days 1-28, daratumumab is administered subcutaneously at a dose of 1800 mg on days 1 and 15, and dexamethasone is administered orally or intravenously at a dose of 40 mg on days 1, 8, 15, and 22; and
during a seventh cycle or longer of 28 days, 400 mg of venetoclax is administered on days 1-28, daratumumab is administered subcutaneously at a dose of 1800 mg on day 1, and dexamethasone is administered orally or intravenously at a dose of 40 mg on days 1, 8, 15, and 22.
